(19) United States
(12) Patent Application Publication
(10) Pub. No.: US 2005/0028302 A1
(43) Pub. Date: Feb. 10, 2005

(54) Dye composition comprising at least one oxidation base, 2-chloro-6-methyl-3-aminophenol and 3-methyl-1-phenyl-5-pyrazolone

(76) Inventors: Marie-Pascale Audousset, Asnieres (FR); Eric Bone, Rueil Malmaison (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/879,184

(22) Filed: Jun. 30, 2004

Related U.S. Application Data
(60) Provisional application No. 60/506,512, filed on Sep. 29, 2003.

(30) Foreign Application Priority Data
Jul. 1, 2003 (FR) 03 07957

(51) Int. Cl. A61K 7/13

(52) U.S. Cl. 8/405

(57) ABSTRACT

The present disclosure relates to a composition for the oxidation dyeing of keratin fibers, for instance human keratin fibers such as the hair, comprising at least one oxidation base, 2-chloro-6-methyl-3-aminophenol and 3-methyl-1-phenyl-5-pyrazolone. The present disclosure also relates to the process of dyeing keratin fibers comprising the application of the composition for the oxidation dyeing of keratin fibers to keratin fibers.

This application claims benefit of U.S. Provisional Application No. 60/506,512, filed Sep. 29, 2003.

The present disclosure relates to a composition for the oxidation dyeing of keratin fibers, for instance, human keratin fibers such as the hair, comprising at least one oxidation base, 2-chloro-6-methyl-3-aminophenol and 3-methyl-1-phenyl-5-pyrazolone.

It is known to dye keratin fibers, for instance human keratin fibers such as the hair, with dye compositions comprising oxidation dye precursors, which are generally known as oxidation bases, such as ortho- or para-phenylenediamines, ortho- or para-aminophenols and heterocyclic compounds. These oxidation bases are colorless or weakly colored compounds that, when combined with oxidizing products, can give rise to colored compounds by a process of oxidative condensation.

It is also known that the shades obtained with these oxidation bases may be varied by combining them with couplers or coloration modifiers, for example, coloration modifiers such as aromatic meta-diamines, meta-aminophenols, meta-diphenols and certain heterocyclic compounds such as indole compounds may be used.

The variety of molecules that can be used as oxidation bases and couplers allows a wide range of colors to be obtained.

The “permanent” coloration obtained with oxidation dyes should be capable of satisfying at least one of a certain number of requirements. For example, ideally the oxidation dyes have no toxicological drawbacks, they allow shades to be obtained in the desired intensity, and/or it they provide a color capable of showing good resistance to external agents such as light, bad weather, washing, permanent-waving, perspiration and rubbing. The dyes may also allow white hairs to be covered in an unselective manner, i.e. to produce the smallest possible color differences along the same length of keratin fiber, that may in fact be differently sensitised, e.g., damaged, between its end and its root.

Compositions for the oxidation dyeing of keratin fibers, comprising 2-chloro-6-methyl-3-aminophenol or 2-methyl-6-chloro-3-aminophenol as a coupler, in combination with oxidation bases conventionally used for oxidation dyeing, for instance certain para-phenylenediamines, para-aminophenol or heterocyclic bases, have already been proposed, for example in German patent application No. DE 30 16 008. Patent application No. WO 96/15765 describes compositions for the oxidation dyeing of keratin fibers, comprising 2-chloro-6-methyl-3-aminophenol as a coupler and 2-(2,5-diaminophenyl)ethanol as an oxidation base. Patent application No. WO 96/15766 describes compositions for the oxidation dyeing of keratin fibers, comprising the specific combination of 2-chloro-6-methyl-3-aminophenol as the coupler and a particular oxidation base chosen from para-aminophenol derivatives substituted in position 2 or 3, for instance 3-methyl-4-aminophenol, 2-allyl-4-aminophenol or 2-aminomethyl-4-aminophenol. French Patent No. FR 1,426,889 describes compositions for the oxidation dyeing of keratin fibers, comprising 3-methyl-1-phenyl-5-pyrazolone as a coupler, in combination with a particular para-phenylenediamine as oxidation base.

However, such compositions are not entirely satisfactory, especially with regard to the staying power of the colorations obtained with respect to the various attacking factors to which the hair may be subjected, for example, shampoo, light, perspiration, and permanent-reshaping operations, and also with regard to the strength, selectivity and chromaticity of the colorations obtained. Furthermore, when it is present in the dye composition, 2-chloro-6-methyl-3-aminophenol is not always stable, which can have the effect of producing unsatisfactory colorations.

Thus, one aspect of the present disclosure is to provide novel compositions for the oxidation dyeing of keratin fibers that do not have the drawbacks of those of the prior part. For instance, one aspect of the present invention is to provide novel compositions that are capable of producing strong, chromatic, aesthetic colorations in varied shades, which are capable of showing little selectivity and good resistance to the various attacking factors to which the hair may be subjected, such as shampoo, light, sweat and permanent-reshaping operations.

Accordingly, one aspect of the present disclosure is a composition for the oxidation dyeing of keratin fibers, comprising, in a suitable dyeing medium:
at least one oxidation base;
at least one coupler chosen from 2-chloro-6-methyl-3-aminophenol and the addition salts thereof; and
3-methyl-1-phenyl-5-pyrazolone and/or addition salts thereof.

The composition according to the present disclosure can make it possible to obtain, for example, a strong, chromatic, aesthetic coloration of keratin fibers that show little selectivity and is resistant with respect to the various attacking factors to which the keratin fibers may be subjected. Furthermore, 2-chloro-6-methyl-3-aminophenol shows good stability in the composition as disclosed herein.

Another aspect of the present disclosure is a process for the oxidation dyeing of keratin fibers, for instance, human keratin fibers such as the hair, using the composition disclosed herein.

Yet another aspect of the present disclosure is the use of the composition according to the present disclosure for the oxidation dyeing of keratin fibers, for instance human keratin fibers, such as the hair.

Still another aspect of the present disclosure is the use of 3-methyl-1-phenyl-5-pyrazolone to stabilize the 2-chloro-6-methyl-3-aminophenol in the compositions.

The oxidation bases that may be used in the dye compositions in accordance with the present disclosure may be chosen from, for example, para-phenylenediamines, double bases, para-aminophenols, ortho-aminophenols, heterocyclic oxidation bases, and the addition salts thereof.

Among the para-phenylenediamines that may be used as oxidation bases in the dye composition in accordance with the present disclosure, non-limiting mention may be made of, for instance, the compounds of formula (I), and the addition salts thereof: 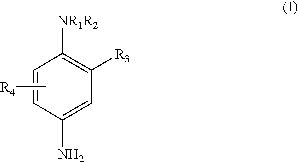
wherein:
R₁ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, C₁-C₄ alkyl radicals substituted with a nitrogenous group, phenyl radicals and 4′-aminophenyl radicals;
R₂ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals and C₁-C₄ alkyl radicals substituted with a nitrogenous group;
R₁ and R₂ may form, together with the nitrogen atom to which they are attached, a 5- to 8-membered heterocycle optionally substituted with at least one radical chosen from C₁-C₄ alkyl, amino, C₁-C₄ hydroxyalkyl and trialkylammonium radicals;
R₃ is chosen from a hydrogen atom, halogens such as chlorine, bromine, iodine or fluorine atoms, C₁-C₄ alkyl radicals, C₁-C₄ monohydroxyalkyl radicals, C₁-C₄ hydroxyalkoxy radicals, acetylamino(C₁-C₄)alkoxy radicals, mesylamino(C₁-C₄)alkoxy radicals and carbamoylamino(C₁-C₄)alkoxy radicals,
R₄ is chosen from hydrogen and halogen atoms, and C₁-C₄ alkyl radicals.

Among the para-phenylenediamines of formula (I) above that may be used, non-limiting mention may be made of, for example, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(p-hydroxyethyl)-para-phenylenediamine, 4-amino-N,N-bis(β-hydroxyethyl)-2-methylaniline, 4-amino-N,N-bis(β-hydroxyethyl)-2-chloroaniline, 2-(β-hydroxyethyl)-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-(β-hydroxyethyloxy)-para-phenylenediamine, 2-(β-acetylaminoethyloxy)-para-phenylenediamine, N-(β-methoxyethyl)-para-phenylenediamine and 2-(β-hydroxyethyl)amino-5-aminotoluene, and the addition salts thereof.

Further among the para-phenylenediamines of formula (I) above, non-limiting mention may be made of para-phenylenediamine, para-tolylenediamine, 2-isopropyl-para-phenylenediamine, 2-(β-hydroxyethyl)-para-phenylenediamine, 2-(β-hydroxyethyloxy)-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-(β-acetylaminoethyloxy)-para-phenylenediamine, and the addition salts thereof.

According to the present disclosure, the term “double bases” means compounds comprising at least two aromatic nuclei bearing groups chosen from amino and hydroxyl groups.

Among the double bases that may be used as oxidation bases in the dye compositions according to the present disclosure, non-limiting mention may be made of, for example, the compounds of formula (II), and the addition salts thereof: 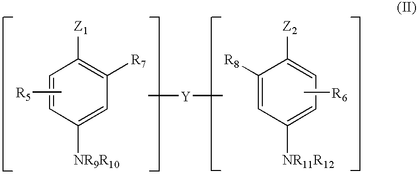
wherein:
Z₁ and Z₂, which may be identical or different, are chosen from hydroxyl and amino radicals that may be substituted with C₁-C₄ alkyl radicals or with a linker arm Y;
the linker arm Y is chosen from linear and branched alkylene chains comprising from 1 to 14 carbon atoms, that may be interrupted by or terminated with at least one entity chosen from nitrogenous groups, and hetero atoms such as oxygen, sulphur or nitrogen atoms, and optionally substituted with at least one radical chosen from hydroxyl and C₁-C₆ alkoxy radicals;
R₅ and R₆ are chosen from hydrogen and halogen atoms, C₁-C₄ alkyl radicals, C₁-C₄ monohydroxyalkyl radicals, C₂-C₄ polyhydroxyalkyl radicals, C₁-C₄ aminoalkyl radicals, and linker arms Y;
R₇, R₈, R₉, R₁₀, R₁₁ and R₁₂, which may be identical or different, are chosen from hydrogen atom, linker arms Y, C₁-C₄ alkyl radicals and C₁-C₄ monohydroxyalkyl radicals; with the proviso that the compounds of formula (II) comprise only one linker arm Y per molecule.

Among the double bases of formula (II) above derived from para-phenylenediamine, non-limiting mention may be made of, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4′-aminophenyl)tetramethylenediamine, N,N′-bis(β-hydroxyethyl)-N,N′-bis(4′-aminophenyl)tetramethylenediamine, N,N′-bis(4′-methylaminophenyl)tetramethylenediamine, N,N′-bis(ethyl)-N,N′-bis(4′-amino-3′-methylphenyl)ethylenediamine, 1,8-bis(2′,5′-diaminophenoxy)-3,6-dioxaoctane, and the addition salts thereof.

Further among the double bases of formula (II) above derived from para-aminophenol, non-limiting mention may be made of 4-amino-6-[(5′-amino-2′-hydroxy-3′-methylphenyl)methyl]-2-methylphenol, bis(5′-amino-2′-hydroxyphenyl)methane, and the addition salts thereof.

In one embodiment of the present disclosure, the double bases of formula (II) are chosen from N,N′-bis(β-hydroxyethyl)-N,N′-bis(4′-aminophenyl)-1,3-diaminopropanol, 1,8-bis(2,5-diaminophenoxy)-3,5-dioxaoctane, and the addition salts thereof.

Among the para-aminophenols that may be used as oxidation bases in the dye compositions according to the present disclosure, non-limiting mention may be made, for example, of the compounds of formula (III), and the addition salts thereof: 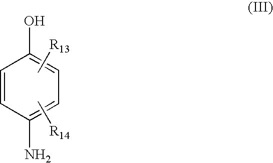
wherein:
R₁₃ is chosen from hydrogen and halogen atoms, C₁-C₄ alkyl, C₁-C₄ monohydroxyalkyl, (C₁-C₄)alkoxy(C₁-C₄)alkyl, C₁-C₄ aminoalkyl and hydroxy(C₁-C₄)alkylamino(C₁-C₄)alkyl radicals;
R₁₄ is chosen from hydrogen and halogen atoms, C₁-C₄-alkyl, C₁-C₄ monohydroxyalkyl, C₂-C₄ polyhydroxyalkyl, C₁-C₄ aminoalkyl, C₁-C₄ cyanoalkyl and (C₁-C₄)alkoxy-(C₁-C₄)alkyl radical.

Among the para-aminophenols of formula (III) above, non-limiting mention may be made of, for example, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2-chlorophenol and 4-amino-2,6-dichlorophenol, and the addition salts thereof.

Among the ortho-aminophenols that may be used as oxidation bases in the dye compositions according to the present disclosure, non-limiting mention may be made of, for example, 2-aminophenol, 2-amino-5-methylphenol, 2-amino-6-methylphenol, 5-acetamido-2-aminophenol, and the addition salts thereof.

Among the heterocyclic bases that may be used as oxidation bases in the dye compositions according to the present disclosure, non-limiting mention may be made of, for example, pyridine derivatives, pyrimidine derivatives, pyrazole derivatives, pyrazolopyrimidine derivatives, and the addition salts thereof.

Among the pyridine derivatives that may be used as disclosed herein, non-limiting mention may be made, for instance, of the compounds described, for example, in British Patent Nos. GB 1,026,978 and GB 1,153,196, as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof.

Other pyridine oxidation bases that are useful in the present invention include the 3-aminopyrazolo[1,5-a]pyridine oxidation bases and the addition salts thereof described, for example, in patent application FR 2,801,308. By way of example, non-limiting mention may be made of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyrid ine-2-carboxylic acid, 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2,3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol, and also the addition salts thereof.

Among the pyrimidine derivatives that may be used as disclosed herein, non-limiting mention may be made of the compounds described, for example, in German Patent No. DE 23 59 399 or Japanese Patent Nos. JP 88-169571 and JP 05-63124; EP 0 770 375 or patent application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, the addition salts thereof and the tautomeric forms thereof, when a tautomeric equilibrium exists.

Among the pyrazole derivatives that may be used as disclosed herein, non-limiting mention may be made of, for example, the compounds described in German Patent Nos. DE 38 43 892 and DE 41 33 957 and patent applications WO 94/08969, WO 94/08970, FR-A-2 733 749 and DE 195 43 988, such as 4,5-diamino-1-methylpyrazole, 4,5-d iamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-d iamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-(β-hydroxyethyl)-3-methylpyrazole, 4,5-diamino-1-tert-butyl-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof.

Among the pyrazolopyrimidine derivatives that may be used according to the present disclosure, non-limiting mention may be made of, for example, the pyrazolo[1,5-a]pyrimidines of formula (IV), and the addition salts thereof with an acid or with a base and the tautomeric forms thereof, when a tautomeric equilibrium exists: 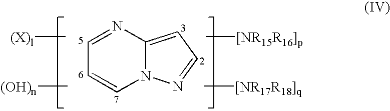
wherein:
R₁₅, R₁₆, R₁₇ and R₁₈, which may be identical or different, are chosen from hydrogen atoms, C₁-C₄ alkyl radicals, aryl radials, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals, wherein the amine is optionally protected with an acetyl, ureido or sulphonyl radical; (C₁-C₄)alkylamino(C₁-C₄)alkyl radicals, di[(C₁-C₄)alkyl]amino(C₁-C₄)alkyl radicals, wherein the dialkyl radicals may optionally form a 5- or 6-membered carbon-based ring or a heterocycle; and hydroxy(C₁-C₄)alkyl- and di[hydroxy-(C₁-C₄)alkyl]amino(C₁-C₄)alkyl radicals;
the radicals X, which may be identical or different, are chosen from hydrogen atoms, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di[(C₁-C₄)alkyl]amino(C₁-C₄)alkyl radicals, wherein the dialkyls may optionally form a 5- or 6-membered carbon-based ring or heterocycle; hydroxy(C₁-C₄)alkyl- and di[hydroxy(C₁-C₄)alkyl]amino(C₁-C₄)alkyl radicals; amino radicals, (C₁-C₄)alkyl- and di[(C₁-C₄)alkyl]amino radicals; halogen atoms, carboxylic acid groups and sulphonic acid groups;
i is equal to 0, 1, 2 or 3;
p is equal to 0 or 1;
q is equal to 0 or 1;
n is equal to 0 or 1;
   with the proviso that:
the sum p+q is other than 0;
when p+q is equal to 2, then n is equal to 0 and the groups NR₁₅R₁₆ and NR₁₇R₁₈ occupy the (2,3); (5,6); (6,7); (3,5) or (3,7) positions;
when p+q is equal to 1, then n is equal to 1 and the group NR₁₅R₁₆ (or NR₁₇R₁₈) and the OH group occupy the (2,3); (5,6); (6,7); (3,5) or (3,7) positions.

When the pyrazolo[1,5-a]pyrimidines of formula (IV) above are such that they comprise a hydroxyl group on one of the positions 2, 5 or 7 a to a nitrogen atom, a tautomeric equilibrium exists represented, for example, by the following scheme:

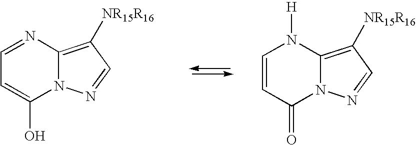

Among the pyrazolo[1,5-a]pyrimidines of formula (IV) above, non-limiting mention may be made of, for example, pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, and the addition salts thereof and the tautomeric forms thereof, when a tautomeric equilibrium exists.

The pyrazolo[1,5-a]pyrimidines of formula (IV) above can be prepared by cyclization starting with an aminopyrazole, according to the syntheses described in the following references:
European Patent No. 628559;
R. Vishdu, H. Navedul, Indian J. Chem., 34b (6), 514, 1995;
N. S. Ibrahim, K. U. Sadek, F. A. Abdel-AI, Arch. Pharm., 320, 240, 1987;
R. H. Springer, M. B. Scholten, D. E. O'Brien, T. Novinson, J. P. Miller, R. K. Robins, J. Med. Chem., 25, 235, 1982;
T. Novinson, R. K. Robins, T. R. Matthews, J. Med. Chem., 20, 296, 1977; and
U.S. Pat. No. 3,907,799.

The pyrazolo[1,5-a]pyrimidines of formula (IV) above can also be prepared by cyclization starting from hydrazine, according to the syntheses described in the following references:
A. McKillop and R. J. Kobilecki, Heterocycles, 6(9),1355, 1977;
E. Alcade, J. De Mendoza, J. M. Marcia-Marquina, C. Almera, J. Elguero, J. Heterocyclic Chem., 11 (3), 423, 1974; and
K. Saito, I. Hori, M. Higarashi, H. Midorikawa, Bull. Chem. Soc. Japan, 47(2), 476, 1974.

The composition according to the present disclosure may also comprise at least one additional conventional coupler chosen from the couplers conventionally used for the oxidation dyeing of keratin fibers. Among such couplers, non-limiting mention may be made of meta-phenylenediamines, meta-aminophenols other than 2-chloro-6-methyl-3-aminophenol, meta-diphenols, naphthalene-based couplers, heterocyclic couplers other than 3-methyl-1-phenyl-5-pyrazolone, and the addition salts thereof.

Non-limiting examples that may further be mentioned include 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-d iamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and the addition salts thereof.

The at least one oxidation base is present in the composition in an amount ranging from 0.001% to 10% by weight for each oxidation base present, for instance, ranging from 0.005% and 6% by weight, relative to the total weight of the dye composition.

In the compositions of the present disclosure, the at least one coupler chosen from 2-chloro-6-methyl-3-aminophenol and the addition salts thereof is present in an amount ranging from 0.001% to 10% by weight, for instance from 0.005% to 6% by weight, relative to the total weight of the dye composition.

The 3-methyl-1-phenyl-5-pyrazolone and/or addition salts thereof, is present in an amount ranging from 0.001% to 10% by weight, such as from 0.005% to 6% by weight, relative to the total weight of the dye composition. Wherein when additional couplers are present, each additional coupler may also be present in an amount ranging from 0.001% to 10% by weight, such as from 0.005% to 6% by weight, relative to the total weight of the dye composition.

For example, the ratio of the weight of the 2-chloro-6-methyl-3-aminophenol and/or addition salts thereof to the weight of the 3-methyl-1-phenyl-5-pyrazolone and/or addition salts thereof can range from 1 to 100.

The addition salts of the compounds that may be used according to the present disclosure, may be chosen from, for example the addition salts with an acid, such as the hydrochlorides, hydrobromides, sulphates, citrates, succinates, tartrates, lactates, tosylates, benzenesulphonates, phosphates and acetates, and the addition salts with a base, such as sodium hydroxide, potassium hydroxide, aqueous ammonia, amines or alkanolamines.

The dye compositions in accordance with the present disclosure may also comprise at least one direct dye that may be chosen, for example, from nitrobenzene dyes, azo direct dyes and methine direct dyes. These direct dyes may be of nonionic, anionic or cationic nature.

The medium that is suitable for dyeing, also known as a dye support, comprises water or a mixture of water and of at least one organic solvent to dissolve the compounds that would not be sufficiently soluble in the water. Non-limiting examples of organic solvents that may be mentioned include C₁-C₄ lower alkanols, such as ethanol and isopropanol; polyols and polyol ethers, for instance 2-butoxyethanol, propylene glycol, propylene glycol monomethyl ether, diethylene glycol monoethyl ether and monomethyl ether, and also aromatic alcohols, for instance benzyl alcohol or phenoxyethanol, and mixtures thereof.

The at least one organic solvent may be present in the composition in an amount ranging from 1% to 40% by weight such as from 5% to 30% by weight, relative to the total weight of the dye composition.

The dye compositions in accordance with the present disclosure may also comprise at least one adjuvant conventionally used in hair dye compositions, such as anionic, cationic, nonionic, amphoteric or zwitterionic surfactants or mixtures thereof; anionic, cationic, nonionic, amphoteric or zwitterionic polymers or mixtures thereof; mineral or organic thickeners, other than anionic, cationic, nonionic and amphoteric associative polymers; antioxidants; penetrating agents; sequestering agents; fragrances; buffers; dispersants; conditioners, for instance volatile or non-volatile, modified or unmodified silicones; film-forming agents; ceramides; preserving agents and opacifiers.

The at least one adjuvant may be present in an amount for each adjuvant ranging from 0.001% to 20% by weight, relative to the total weight of the composition.

Needless to say, a person skilled in the art will take care to select any of these additional optional compounds such that the advantageous properties intrinsically associated with the oxidation dye composition in accordance with the present disclosure are not, or are not substantially, adversely affected by the envisaged additions.

The pH of the dye compositions in accordance with the present disclosure ranges from 3 to 12 for instance, from 5 to 11. It may be adjusted to the desired value by means of acidifying or basifying agents usually used for dyeing keratin fibers, or alternatively using standard buffer systems.

Among the acidifying agents that may be used as disclosed herein, non-limiting mention may be made, for example, of mineral or organic acids, for instance hydrochloric acid, orthophosphoric acid, sulphuric acid, carboxylic acids, for instance acetic acid, tartaric acid, citric acid or lactic acid, and sulphonic acids.

Among the basifying agents that may be used as disclosed herein, non-limiting mention may be made, for example, of aqueous ammonia, alkaline carbonates, alkanolamines such as monoethanolamine, diethanolamine and triethanolamine and derivatives thereof, sodium hydroxide, potassium hydroxide and the compounds of formula (XVI): 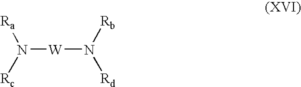
wherein W is a propylene residue optionally substituted with a hydroxyl group or a C₁-C₄ alkyl radical; Rₐ, R_{b}, R_{c} and R_{d}, which may be identical or different, are chosen from hydrogen atoms, C₁-C₄ alkyl and C₁-C₄ hydroxyalkyl radicals.

The dye compositions according to the present disclosure may be in various forms, such as in the form of liquids, creams or gels, or in any other form that is suitable for dyeing keratin fibers, for instance, human hair.

The process of the present disclosure is a process in which the compositions according to the present disclosure as defined above is applied to keratin fibers, and the color is developed using at least one oxidizing agent. The color may be revealed at acidic, neutral or alkaline pH and the at least one oxidizing agent may be added to the dye compositions as disclosed herein just at the time of use, or it may be introduced using an oxidizing composition comprising it, applied simultaneously with or sequentially to the composition as disclosed herein.

According to one embodiment of the present disclosure, for example, the composition as disclosed herein is mixed, for instance at the time of use, with a composition comprising, in a medium that is suitable for dyeing, at least one oxidizing agent, wherein the at least one oxidizing agent is present in an amount sufficient to develop a coloration. The mixture obtained is then applied to keratin fibers. After a leave-in time ranging from 3 to 50 minutes, such as from 5 to 30 minutes, the keratin fibers are rinsed, washed with shampoo, rinsed again and then dried.

The oxidizing agents conventionally used for the oxidation dyeing of keratin fibers, and which may be used as disclosed herein, include, for example, hydrogen peroxide, urea peroxide, alkali metal bromates, persalts such as perborates and persulphates, peracids, and oxidase enzymes, among which non-limiting mention may be made of peroxidases, 2-electron oxidoreductases such as uricases, and 4-electron oxygenases, for instance laccases. Hydrogen peroxide is particularly preferred.

The oxidizing composition may also comprise various adjuvants conventionally used in hair dye compositions and as defined above.

The pH of the oxidizing composition comprising the at least one oxidizing agent is such that, after mixing with the dye composition, the pH of the resulting composition applied to the keratin fibers can range from 3 to 12, for instance, from 5 to 11. It may be adjusted to the desired value by means of acidifying or basifying agents usually used in the dyeing of keratin fibers and as defined above.

The ready-to-use composition that is finally applied to keratin fibers may be in various forms, such as in the form of liquids, creams or gels, or in any other form that is suitable for dyeing keratin fibers, and especially human hair.

Another aspect of the present disclosure is also a multi-compartment device or “kit” for dyeing, wherein at least one first compartment comprises a dye composition in accordance with the present disclosure and at least one second compartment comprises an oxidizing composition. This device may be equipped with a means for applying the desired mixture to the hair, such as the devices described in French Patent No. FR 2,586,913.

Using this device, it is possible to dye keratin fibers via a process that involves mixing a dye composition as disclosed herein with at least one oxidizing agent as defined above, and applying the mixture obtained to keratin fibers for a time that is sufficient to develop the desired coloration.

Another aspect of the present disclosure is the use of 3-methyl-1-phenyl-5-pyrazolone and/or the addition salts thereof to stabilize 2-chloro-6-methyl-3-aminophenol and/or the addition salts thereof as a solution.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The examples that follow serve to illustrate the invention without, however, being limiting in nature.

### EXAMPLES

The dye compositions below were prepared:

| | Example | | | |
|---|---|---|---|---|
| | 1 | 2 | 3 | 4 |
| 2-Chloro-6-methyl-3-aminophenol | 1.26 g | 1.26 g | 1.26 g | 1.26 g |
| para-Aminophenol | 0.872 g | | | |
| 3-Methyl-4-aminophenol | | | | 0.984 g |
| para-Phenylenediamine | | | 0.864 g | |
| para-Tolylenediamine | | 0.976 g | | |
| 3-Methyl-1-phenyl-5-pyrazolone | 0.15 g | 0.15 g | 0.15 g | 0.15 g |
| Dye support* | (*) | (*) | (*) | (*) |
| Demineralized water qs | 100 g | 100 g | 100 g | 100 g |

(*) Common dye support:

| | | |
|---|---|---|
| Oleyl alcohol polyglycerolated with 2 mol of glycerol | 4 | g |
| Oleyl alcohol polyglycerolated with 4 mol of glycerol, | 5.69 | g A.M. |
| containing 78% active material (A.M.) | | |
| Oleic acid | 3.0 | g |
| Oleylamine containing 2 mol of ethylene oxide, sold | 7 | g |
| under the trade name Ethomeen O12 by the company | | |
| Akzo | | |
| Diethylaminopropyl laurylaminosuccinamate, sodium salt | 3.0 | g |
| containing 55% A.M. | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9 | g |
| Sodium metabisulphite as an aqueous solution | 0.455 | g A.M. |
| containing 35% A.M. | | |
| Ammonium acetate | 0.8 | g |
| Antioxidant, sequestering agent | qs | |
| Fragrance, preserving agent | qs | |
| Aqueous ammonia containing 20% NH₃ | 10 | g |

The dye composition was mixed extemporaneously with its equivalent weight amount of 20-volumes aqueous hydrogen peroxide solution.

The mixture thus prepared was applied to locks of natural grey hair containing 90% white hairs, at a rate of 28 g per 3 g of hair, for 30 minutes. The hair was then rinsed, washed with a standard shampoo and dried.

The hair coloration was evaluated visually. The shades obtained are given in the table below.

| | Example | | | |
|---|---|---|---|---|
| | 1 | 2 | 3 | 4 |
| Tone | Blond | Dark | Dark blond | Blond |
| height | | blond | | |
| Glint | Coppery | Purplish | Dark | Red |
| | red | | purple | |

1. A composition for the oxidation dyeing of keratin fibers, comprising, in a suitable dyeing medium:
at least one oxidation base;
at least one coupler chosen from 2-chloro-6-methyl-3-aminophenol and the addition salts thereof; and
3-methyl-1-phenyl-5-pyrazolone and/or the addition salts thereof.

2. The composition according to claim 1, wherein the at least one oxidation base is chosen from para-phenylenediamines, double bases, para-aminophenols, orthoaminophenols, heterocyclic oxidation bases, and the addition salts thereof.

3. The composition according to claim 2, wherein the para-phenylenediamines are chosen from the compounds of formula (I), and the addition salts thereof: 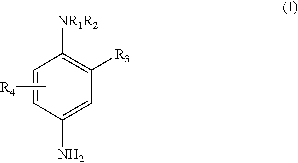
wherein:
R₁ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, C₁-C₄ alkyl radicals substituted with a nitrogenous group, phenyl radicals, 4′-aminophenyl radicals;
R₂ is chosen from a hydrogen atom, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals and C₁-C₄ alkyl radicals substituted with a nitrogenous group;
R₁ and R₂ may form together, with the nitrogen atom to which they are attached, a 5- to 8-membered heterocycle optionally substituted with at least one radical chosen from C₁-C₄ alkyl radicals, amino radicals, C₁-C₄ hydroxyalkyl radicals and trialkylammonium radicals;
R₃ is chosen from hydrogen and halogen atoms, C₁-C₄ alkyl radicals, C₁-C₄ monohydroxyalkyl radicals, C₁-C₄ hydroxyalkoxy radicals, acetylamino(C₁-C₄)alkoxy radicals, mesylamino(C₁-C₄)alkoxy radicals and carbamoylamino(C₁-C₄)alkoxy radicals; and
R₄ is chosen from hydrogen and halogen atoms, and C₁-C₄ alkyl radicals.

4. The composition according to claim 3, wherein the halogen atoms are chosen from chlorine, bromine, iodine and fluorine atoms.

5. The composition according to claim 3, in which the para-phenylenediamines of formula (I) are chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylanili ne, 4-amino-N,N-bis(β-hydroxyethyl)-2-chloroaniline, 2-(β-hydroxyethyl)-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-(β-hydroxyethyloxy)-para-phenylenediamine, 2-(β-acetylaminoethyloxy)-para-phenylenediamine, N-amino-(β-methoxyethyl)-para-phenylenediamine and the addition salts thereof.

6. The composition according to claim 2, in which the double bases are chosen from the compounds of formula (II), and the addition salts thereof: 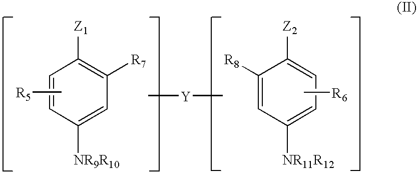
wherein:
Z₁ and Z₂, which may be identical or different, are chosen from hydroxyl and amino radicals that may be substituted with a C₁-C₄ alkyl radical or with a linker arm Y;
the linker arm Y is chosen from linear and branched alkylene chains comprising from 1 to 14 carbon atoms, that may be interrupted by or terminated with at least one entity chosen from nitrogenous groups and hetero atoms, and optionally substituted with at least one radical chosen from hydroxyl and C₁-C₆ alkoxy radicals;
R₅ and R₆ are chosen from hydrogen and halogen atoms, C₁-C₄ alkyl radicals, C₁-C₄ monohydroxyalkyl radicals, C₂-C₄ polyhydroxyalkyl radicals, C₁-C₄ aminoalkyl radicals, or a linker arm Y;
R₇, R₈, R₉, R₁₀, R₁₁ and R₁₂, which may be identical or different, are chosen from hydrogen atoms, C₁-C₄ alkyl radicals, C₁-C₄ monohydroxyalkyl radicals, and a linker arm Y; with the proviso that the compounds of formula (II) comprise only one linker arm Y per molecule.

7. The composition according to claim 6, wherein the hetero atoms are chosen from oxygen, sulfur and nitrogen atoms.

8. The composition according to claim 6, wherein the double bases of formula (II) are chosen from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, 4-amino-6-[(5′-amino-2′-hydroxy-3′-methylphenyl)methyl]-2-methylphenol, bis(5-amino-2-hydroxyphenyl)methane, and the addition salts thereof.

9. The composition according to claim 2, wherein the para-aminophenols are chosen from the compounds of formula (III), and the addition salts thereof: 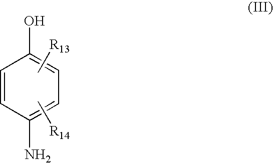
wherein:
R₁₃ is chosen from a hydrogen atom, a halogen atom, C₁-C₄ alkyl radicals, C₁-C₄ monohydroxyalkyl radicals, (C₁-C₄)alkoxy(C₁-C₄)alkyl radicals, C₁-C₄ aminoalkyl radicals and hydroxy(C₁-C₄)alkylamino(C₁-C₄)alkyl radicals;
R₁₄ is chosen from a hydrogen atom, a halogen atom, C₁-C₄-alkyl radicals, C₁-C₄ monohydroxyalkyl radicals, C₂-C₄ polyhydroxyalkyl radicals, C₁-C₄ aminoalkyl radicals, C₁-C₄ cyanoalkyl radicals and (C₁-C₄)alkoxy(C₁-C₄)alkyl radicals;
with the proviso that at least one of the radicals R₁₃ and R₁₄ is a hydrogen atom.

10. The composition according to claim 9, wherein the para-aminophenols are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2-chlorophenol, 4-amino-2,6-dichlorophenol, and the addition salts thereof.

11. The composition according to claim 2, wherein the ortho-aminophenols are chosen from 2-aminophenol, 2-amino-5-methylphenol, 2-amino-6-methylphenol, 5-acetamido-2-aminophenol, and the addition salts thereof.

12. The composition according to claim 2, wherein the heterocyclic bases are chosen from pyridine derivatives, pyrimidine derivatives, pyrazole derivatives, pyrazolopyrimidine derivatives, and the addition salts thereof.

13. The composition according to claim 1, further comprising at least one additional coupler chosen from meta-phenylenediamines, meta-aminophenols other than 2-chloro-6-methyl-3-aminophenol, meta-diphenols, naphthalene-based couplers, heterocyclic couplers other than 3-methyl-1-phenyl-5-pyrazolone, and the addition salts thereof.

14. The composition according to claim 1, wherein the at least one oxidation base is present in an amount ranging from 0.001% to 10% by weight, for each oxidation base present, relative to the total weight of the dye composition.

15. The composition according to claim 1, wherein the at least one coupler chosen from 2-chloro-6-methyl-3-aminophenol and the addition salts thereof is present in an amount ranging from 0.001% to 10% by weight, relative to the total weight of the dye composition.

16. The composition according to claim 13, wherein said at least one additional coupler is present in an amount ranging from 0.001% to 10% by weight, for each coupler present, relative to the total weight of the dye composition.

17. The composition according to claim 1, wherein the 3-methyl-1-phenyl-5-pyrazolone and/or the addition salts thereof is present in an amount ranging from 0.001% to 10% by weight, relative to the total weight of the dye composition.

18. The composition according to claim 1, wherein the ratio of the weight of the 2-chloro-6-methyl-3-aminophenol and/or the addition salts thereof to the weight of the 3-methyl-1-phenyl-5-pyrazolone and/or the addition salts thereof ranges from 1 to 100.

19. The composition according to claim 1, further comprising at least one oxidizing agent.

20. The composition according to claim 19, wherein the at least one oxidizing agent is chosen from hydrogen peroxide, urea peroxide, alkali metal bromates, persalts, peracids and oxidase enzymes.

21. A process for the oxidation dyeing of keratin fibers, comprising applying to keratin fibers a dye composition comprising, in a suitable dyeing medium:
at least one oxidation base;
at least one coupler chosen from 2-chloro-6-methyl-3-aminophenol and the addition salts thereof; and
3-methyl-1-phenyl-5-pyrazolone and/or addition salts thereof; and
developing the color with at least one oxidizing agent.

22. The process according to claim 21, wherein the at least one oxidizing agent is chosen from hydrogen peroxide, urea peroxide, alkali metal bromates, persalts, peracids and oxidase enzymes.

23. The process according to claim 21, wherein the at least one oxidizing agent is mixed with the dye composition at the time of application.

24. The process according to claim 21, wherein a composition comprising the at least one oxidizing agent is applied to the keratin fibers simultaneously with or sequentially to the dye composition.

25. A process for dyeing keratin fibers comprising,
mixing a dye composition comprising, in a suitable dyeing medium,
at least one oxidation base,
at least one coupler chosen from 2-chloro-6-methyl-3-aminophenol and the addition salts thereof, and
3-methyl-1-phenyl-5-pyrazolone and/or addition salts thereof,
with at least one oxidizing agent present in an amount sufficient to develop coloration, to create a ready-to-use composition;
applying this ready-to-use-composition to keratin fibers;
leaving the composition on the keratin fibers for a period of time ranging from 3 to 50 minutes;
rinsing the keratin fibers, washing the keratin fibers with shampoo, rinsing the fibers again and then drying the fibers.

26. The process according to claim 25, wherein the ready-to-use composition is created on the keratin fibers.

27. A multi-compartment kit, comprising
at least one first compartment comprising a dye composition comprising, in a suitable dyeing medium,
at least one oxidation base;
at least one coupler chosen from 2-chloro-6-methyl-3-aminophenol and the addition salts thereof; and
3-methyl-1-phenyl-5-pyrazolone and/or addition salts thereof; and
at least one second compartment comprising a composition comprising at least one oxidizing agent.

28. A process for stabilizing a composition for the oxidation dyeing of keratin fibers, said process comprising:
adding 3-methyl-1-phenyl-5-pyrazolone and/or addition salts thereof to the dyeing composition, wherein said composition comprises, in a suitable dyeing medium, at least one oxidation base and at least one coupler chosen from 2-chloro-6-methyl-3-aminophenol and the addition salts thereof.
